(19) United States
(12) Patent Application Publication
(10) Pub. No.: US 2018/0310658 A1
(43) Pub. Date: Nov. 1, 2018

(54) WEARABLE FOOTWEAR DEGRADATION SENSOR

(71) Applicant: 3M INNOVATIVE PROPERTIES COMPANY, St. Paul, MN (US)

(72) Inventors: Andrew P. Bonifas, Alberta (CA); Nicholas G. Amell, Burnsville, MN (US); Ronald D. Jesme, Plymouth, MN (US); Brock A. Hable, Woodbury, MN (US); Nicholas T. Gabriel, Woodbury, MN (US); Erik K. Iverson, St. Paul, MN (US); Kristy A. Jost, Woodbury, MN (US)

(73) Assignee: 3M INNOVATIVE PROPERTIES COMPANY, St. Paul, MN (US)

(21) Appl. No.: 15/769,239

(22) PCT Filed: Oct. 11, 2016
(86) PCT No.: PCT/US16/56392

§ 371 Date: Apr. 18, 2018

Related U.S. Application Data
(60) Provisional application No. 62/245,034, filed on Oct. 22, 2015.

(51) Int. Cl. A43B 3/00; G01D 5/243; A43B 5/06; A61B 5/103

(52) U.S. Cl. CPC A43B 3/0005; G01D 5/243; A43B 17/00; A61B 5/1038; A43B 5/06

(57) ABSTRACT

A shoe (606) degradation sensor (1012, 608) assembly includes a first sensor (1002) disposed in or proximate to a material layer of a shoe (606) between a foot space and an outer surface of the shoe (606). The material layer changes in at least one physical property with degradation to the shoe (606), and the first sensor (1002) is configured to indicate the changing physical property of the material layer thereby indicating a degree of degradation to the shoe (606).

### TECHNICAL FIELD

The invention relates generally to footwear, and more specifically to wearable footwear degradation sensors.

### BACKGROUND

Modern footwear is typically designed to meet several goals, based on factors such as the intended use of the shoe and a desired cost of the shoe. In a typical example, a shoe might be designed to be as light as is practical for its particular use, provide sufficient traction under a variety of conditions, and protect the foot of the wearer from the ground. The shoe may further be designed to provide other functions, such as to protect a user from rain or cold, present a stylish appearance suitable for a particular activity, or protect an athlete from physiological risks associated with various activities.

Running shoes, for example, are typically designed to be lightweight to enhance a runner's speed, while providing good ventilation for the user's foot through use of breathable fabrics in constructing the upper portion of the shoe. The lower portion of the running shoe, or sole, typically provides good traction for athletic movement, while also providing cushioning to lessen the effects of the user's foot repeatedly striking the ground. Modern running shoes often have soles made up of a variety of components to achieve these goals, including a footbed on which the foot sits, an insole under the footbed used to attach the sole to the shoe's upper, a midsole made up of a cushioning material such as a polymeric foam designed to cushion impact resulting from running, and an outsole of a harder rubber material designed to provide good traction with the running surface while providing long tread life.

Each of these components of a running shoe's sole is also constructed with various design goals and tradeoffs taken into consideration. The footbed, for example, may be designed to control moisture or odor, provide cushioning, provide arch support or other position control, or perform other functions depending on the user's particular needs. The outsole may be designed to provide good grip, such as through use of rubber having good frictional properties or spikes, while providing a long wear life. The midsole may similarly be designed to provide cushioning, provide a specific rise from the toe to the heel of the shoe, and provide stability for the user's foot, all while providing a long useful life for the wearer.

In the midsole, the material absorbs 2-3 times the user's body weight during impact in a typical stride, with many hundreds of such impacts per mile run. The midsole material is therefore typically formed of a material that can provide cushioning under repeated high impacts over a long period of time, such as EVA (ethylene vinyl acetate) or PU (polyurethane) foams. Although there are tradeoffs between the various materials used for midsole construction, most midsole materials that provide good cushioning also undergo varying degrees of what is termed “compression set” or flattening with repeated use. For example, EVA foams provide good cushioning and rebound but are somewhat prone to compression set, while PU foams are somewhat more resistant to compression set but provide less cushioning and rebound and are heavier.

Because the cushioning and rebound properties of the shoe often degrade before there is significant wear to the shoe's upper, a pair of shoes may not appear to be worn out despite having significant compression set and loss of cushioning and rebound. Many running shoe users therefore try to estimate when their shoes have lost enough of their ability to provide cushioning or rebound to warrant replacing the shoe with new shoes. This is often done by tracking the number of miles run in a particular pair of shoes, how many weeks or months a particular pair of shoes has been used, or other such methods.

But, such methods don't account for variations in user weight, running surface, stride, or other factors that can significantly affect the effective life of the shoe. Such rules-of-thumb also don't account for differences between shoe materials, midsole thicknesses, or other characteristics of different shoes that contribute to variations in their useful lives.

Because the ability of a running shoe to provide cushioning and rebound to a runner are important factors in the shoe's ability to protect the runner from injury and provide an enjoyable running experience, it is desirable to more accurately determine degradation such as compression set in shoes.

### SUMMARY

One example embodiment of the invention comprises a shoe degradation sensor assembly where a first sensor is disposed in or proximate to a material layer of a shoe between a foot space and an outer surface of the shoe. The material layer changes in at least one physical property with degradation to the shoe, and the first sensor is configured to indicate the physical property of the material layer thereby indicating a degree of degradation to the shoe.

In a further example, a shoe degradation measurement reader comprises an electronic device configured to query a first sensor disposed in or proximate to a material layer of a shoe between a foot space and an outer surface of the shoe, where the material layer changes in at least one physical property with degradation to the shoe. The first sensor is configured to indicate the at least one physical property of the material layer, thereby indicating a degree of degradation to the shoe.

In another example, a shoe degradation measurement system includes a shoe having a first sensor embedded in or proximate to a material layer of the shoe between a foot space and an outer surface of the shoe. The material layer changes in at least one physical property with degradation to the shoe, and the first sensor is configured to indicate the physical property of the material layer thereby indicating a degree of degradation to the shoe. A computerized system is configured to provide an indication of shoe degradation to a user based on measurement of the at least one physical property of the material from the first sensor, and an interface is coupled to the computerized system and is configured to receive from the first sensor an indication of the measurement of the at least one physical property of the material from the first sensor.

The details of one or more examples of the invention are set forth in the accompanying drawings and the description below. Other features and advantages will be apparent from the description and drawings, and from the claims.

### BRIEF DESCRIPTION OF THE FIGURES

FIG. 1 shows a running shoe incorporating a degradation sensor.

FIG. 2 shows an alternate running shoe incorporating a degradation sensor.

FIG. 3 illustrates degradation sensors incorporated into a removable shoe footbed.

FIG. 4 shows an LC tag degradation sensor.

FIG. 5 is a chart illustrating how the resonant frequency of an example LC tag degradation sensor varies with compression set.

FIG. 6 shows an example system for reading the resonant frequency of an LC tag to determine compression set in a shoe.

FIG. 7 shows an example translator device circuit operable to query a degradation sensor and provide an indication of the query result to a user device.

FIG. 8 shows a kiosk incorporating a footwear degradation sensor.

FIG. 9 is a flowchart of a method of using a store kiosk to recommend a replacement shoe to a user.

FIG. 10A shows a running shoe incorporating a degradation sensor.

FIG. 10B shows a running shoe incorporating a degradation sensor and a reader.

FIG. 11 shows a water content shoe degradation sensor.

FIG. 12 shows a method of reading a shoe degradation sensor.

FIG. 13 shows a computerized shoe degradation sensor measurement system.

### DETAILED DESCRIPTION

In the following detailed description of example embodiments, reference is made to specific example embodiments by way of drawings and illustrations. These examples are described in sufficient detail to enable those skilled in the art to practice what is described, and serve to illustrate how elements of these examples may be applied to various purposes or embodiments. Other embodiments exist, and logical, mechanical, electrical, and other changes may be made.

Features or limitations of various embodiments described herein, however important to the example embodiments in which they are incorporated, do not limit other embodiments, and any reference to the elements, operation, and application of the examples serve only to define these example embodiments. Features or elements shown in various examples described herein can be combined in ways other than shown in the examples, and any such combinations is explicitly contemplated to be within the scope of the examples presented here. The following detailed description does not, therefore, limit the scope of what is claimed.

Footwear such as athletic shoes are often constructed not only to protect the foot from contact with the ground, but to provide support and cushioning to the foot to enhance a user's ability to perform various tasks such as running, jumping, and moving with agility. The outsole of such a shoe is typically constructed to provide grip with a particular surface, such as a gym floor or an outdoor track. The midsole is similarly constructed to provide support for lateral movement, cushioning for running or jumping movements, and may provide other features specific to the shoe's application. Because the effectiveness of such features can diminish as the shoe material degrades, it is desirable to ensure that degradation of shoes in ways that might affect various performance characteristics can be monitored and measured.

A running shoe, for example, typically absorbs 2-3 times the user's body weight during impact in a typical stride, with many hundreds of such impacts per mile run. The shoe's midsole undergoes some degradation with each impact, based on factors such as the running surface, the user's stride, the user's weight, and the size of the shoe. This results in “compression set” or flattening of the cushioning material used to construct the midsole, reducing the material's ability to cushion the impact of each running stride. Because high-performance footwear such as running shoes often undergo significant degradation of the midsole and a corresponding reduction in cushioning performance before there are visible signs of wear to the shoe, it is often difficult to estimate when a shoe should be replaced. Methods such as estimation of the number of miles run in a particular pair of shoes are the predominant method of estimating the useful life left in a running shoe, but such methods generally don't account for variations in stride between users, variations in user weight, shoe size relative to the user's weight, or other factors that can significantly affect the rate at which degradation such as compression set of the midsole occur. Further, different shoes may have significantly different degradation characteristics, such as where different materials are used in the outsole, or different thicknesses of various materials are used in constructing different shoe models.

Some examples described herein therefore provide for improved measurement, estimation, or characterization of degradation of material in a shoe, such as measuring compression set in a shoe's midsole by measuring one or more physical properties of the midsole that are related to degradation of the midsole material. In one such example, the distance between an LC resonator and a conductive element disposed in or on the midsole is determined by measuring a resonant property of the LC resonator using an external measurement device. In other examples, other such changes in physical properties of a material, such as a midsole, insole, or padding in the shoe's upper, are measured using other methods.

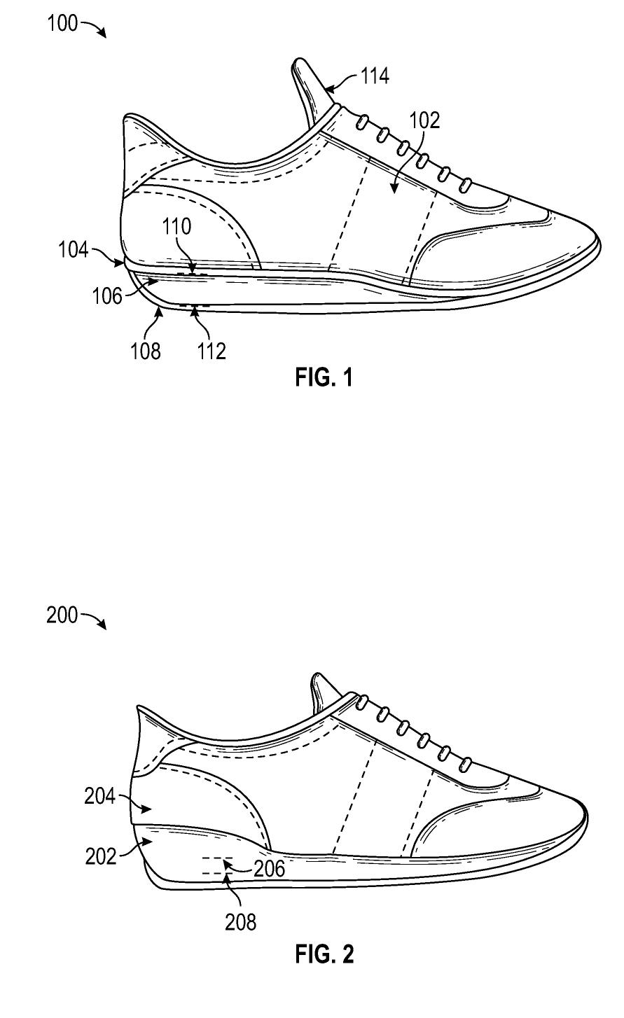

FIG. 1 shows a running shoe incorporating a degradation sensor. Here, a running shoe shown generally at 100 is constructed of an upper 102 that is constructed to contain a user's foot, and a sole made up of an insole 104, a midsole or wedge 106, and an outsole 108. The insole 104, the midsole 106, and the outsole 108 are separate layers, made of separate materials, and are attached to one another such as with an adhesive to form the sole of the shoe. The insole 104 attaches the sole to the shoe upper, while the midsole 106 provides cushioning and elevates the heel slightly above the toe when the user is wearing the shoe. The outsole 108 is made of a rubber material that is harder than the midsole, and provides traction and long wear life for the shoe.

In this example, a degradation sensor is also integrated into the shoe, such as an LC tag 110 that is operable to resonate at a specific frequency and with a specific quality factor (Q) when energized by an external RF energy source. The LC tag in this example is attached to the outer surface of the midsole 106 before the midsole is attached to the insole 104, but in other examples will be otherwise disposed near or in the midsole 106. A conductive element 112 is similarly disposed on the outer surface of the midsole 106 before the midsole is attached to the outsole 108, thereby embedding the conductive element 112 between the midsole and the outsole when the midsole and outsole are attached. The conductive element 112 is in various examples electrically conductive, magnetically conductive, or electrically and magnetically conductive.

As the midsole 106 material degrades, such as by repeated compression as a result of a user running, by heat, by age, and by other such factors, various physical characteristics of the midsole are likely to change in a measurable way. For example, the midsole 106 in this example undergoes a flattening or compression set as a result of the repeated impacts, and is no longer able to fully rebound or recover to its original shape. This change in the midsole material affects the distance between the LC tag 110 and the conductive element 112, causing the LC tag 110 to change in resonant frequency and quality factor, and potentially in other measurable characteristics.

The LC tag 110 can therefore be energized such as using an RF reader device, and the resonant frequency or other resonant characteristic of the LC tag measured, to provide an indication of the distance between the LC tag and the conductive element 112. This indication can then be compared to a reference or expected indicated value to determine whether a maximum allowable degree of compression set is detected, thereby indicating that the shoe is not performing within established performance guidelines and should be replaced. In one such example, the LC tag resonant frequency is compared to a target resonant frequency for the particular model of shoe, based on knowledge regarding the shoe model's initial geometry, materials, and performance guidelines. In another example, a baseline indication is taken for each shoe when new and is recorded, such that the baseline LC tag resonant frequency can be compared against LC tag resonant frequency measurements taken after the shoe has been used, to indicate the degree of change or compression set in the shoe since it was new.

In another example, another resonant characteristic of the LC tag 110, such as the quality factor (Q) or other resonant characteristic of the LC tag is measured, and is used to indicate a change in distance between the LC tag and the conductive element 112. The quality factor of a resonant circuit such as an LC tag is derived from the frequency breadth or bandwidth over which the tag resonates relative to its center resonant frequency. As the LC tag 110 of FIG. 1 moves closer to the conductive element 112, the Q of the circuit will decrease as the resonance bandwidth increases, providing an indication of the distance between the LC tag and the conductive element. The LC tag in these examples is a passive device in that it does not provide power or provide power gain, resulting a reduced cost over typical active devices such as transistors, integrated circuits, and other semiconductor devices.

The LC tag 110 and the conductive element 112 are in this example placed on opposite sides of the midsole 106 foam layer, such that the distance between the LC tag and the conductive element reflects the thickness of the entire layer at a desired location within the shoe, such as under the user's heel. The heel location for the LC tag and conductive element is chosen in this example because the midsole typically experiences the greatest forces directly under the heel, and the midsole is typically therefore thickest under the heel. Degradation of the midsole under the heel will therefore also have the greatest impact on perceived cushioning ability to a user, making the heel a good location for degradation measurement. In other examples, the LC tag, the conductive element, or both may be positioned embedded within a layer, embedded in different layers, sandwiched between different layers, or otherwise configured to measure part of a layer, all of a layer, or multiple layers of a shoe.

In one such example, a shoe comprises layers of EVA (ethylene vinyl acetate) and polyurethane in the midsole, such that the more dense polyurethane foam materials are used to provide structure and support around the heel and arch and the relatively softer EVA foam is used to provide cushioning and rebound. The EVA foam layer is more prone to compression set, but provides significantly better cushioning and rebound or energy storage characteristics than the polyurethane foam. Some examples therefore will use a degradation sensor such as LC tag 110 and conductive element 112 to measure compression in one layer of foam, such as the EVA layer, while not measuring another layer, such as a polyurethane layer, based on the different characteristics of the layers used to form the shoe's sole. In other examples, other layers and materials such as composites, cloths, and the like may be included in shoe construction, and included or excluded from degradation sensing. A shoe incorporating a gel layer, for example, may use the gel layer to provide cushioning such that its thickness is an important indicator of shoe performance, and may elect to measure or not measure the thickness of the gel layer depending on whether construction of the shoe results in the gel layer becoming thinner or otherwise degrading with wear to the shoe. In other examples, the shoe material comprises ethylene vinyl acetate, polyurethane, polymeric foam, rubber, nylon, fabric, gel, adhesive, polychloroprene, thermoplastic resin, thermoset resin, or air, or a combination including two or more of these elements.

Although the example illustrated in FIG. 1 measures compression set in a midsole foam material that is primarily responsible for providing cushioning and rebound to a user while running, other examples include measurement of degradation of different parts of the shoe. A cushioned shoe tongue 114, for example, can also incorporate a degradation sensor such as the LC tag 110 and conductive element 112 on opposite sides of the shoe tongue's foam cushioning material, such that measurement of the resonance of the LC tag indicates a degree of compression set or other degradation of the tongue's foam cushioning material. Degradation sensors are similarly used to measure material degradation in other parts of the shoe in other examples.

Degradation is determined by an observed degree of compression set in the foam midsole 106 in FIG. 1, which is characterized as the degree to which the midsole 106 material does not fully rebound to its original size. For example, a foam midsole that is 0.5 inches thick when new, but which is now 0.4 inches thick, has experienced 20% compression set because it has lost 20% of its thickness due to repeated compressions. In this example, compression set is measured by determining the resonant frequency of LC tag 110, because the resonant frequency of the LC tag varies with the distance between the LC tag 110 and conductive element 112 such that as the midsole experiences compression set and the LC tag becomes nearer to the conductive element 112 with no force applied to the shoe, the resonant frequency of the LC tag increases. In a more detailed example, an LC tag has a resonant frequency of 10.25 MHz in a shoe configuration with no compression set such as that of FIG. 1. When the shoe's midsole is degraded to the point where it has 20% compression set, the LC tag will have a resonant frequency of 10.5 MHz, with frequency change and compression set varying relatively linearly between 0-20% compression set. If the shoe's useful life is considered to be 20% or less compression set, an observed LC tag resonant frequency of 10.5 MHz or greater would therefore indicate that the shoe's useful life has passed. Similarly, a resonant frequency between 10.25-10.5 MHz can indicate the degree of compression set the shoe has already experienced, such as where a user orders a new pair of shoes when the resonant frequency reaches 10.45 MHz, indicating the shoe is nearly worn out.

Because the degree of degradation or compression set in the example of FIG. 1 is determined based on a change in resonance of an LC tag from an expected or reference resonant characteristic, it is desirable in some examples to provide or store baseline LC tag resonance information with which to estimate a shoe's degradation. The LC tag in some such examples incorporates or is otherwise associated with an RFID tag, a passive near-field communications (NFC) tag, or other tag that is operable to store or convey information. In one such example, an RFID or NFC tag contains a serial number or other identifying information for the shoe, which is associated with initial LC tag resonance information. The associated LC tag resonance information may be alternatively stored in the shoe, such as by writing to an NFC tag or RFID embedded in the shoe. This enables storage of baseline information representing the measured physical characteristic or characteristics of each shoe, making determining the degree of degradation of the shoe by monitoring changes in the physical characteristic of the shoe more accurate.

Some examples described herein use radio frequency (RF) devices and signals for purposes such as communication, sensing, power harvesting, and the like. Such radio frequency devices include a broad range of alternatives using electromagnetic energy at various frequencies and using various protocols to achieve the described functions. Near-Field Communication, for example, is a wireless radio frequency signaling protocol used in some examples described herein, but such examples will work equally well with other electromagnetic communication protocols, frequencies, and signaling methods, and are alternative embodiments of those examples. The sensor of FIG. 1 is readable using a radio frequency (RF) signal sweeping a range of anticipated resonant frequencies of the LC tag, but in other embodiments will be electrically readable using other electromagnetic methods, such as other RF signals, galvanic or conductive coupling, or other such use of electric or electromagnetic signals.

FIG. 2 shows an alternate running shoe incorporating a degradation sensor. Here, a running shoe 200 has a heel pocket 204 that is partially supported by a compression molded EVA midsole 202. Compression molding EVA material in a pressurized mold to form the midsole creates a thick skin on the outer surfaces of the midsole, which is more durable than EVA material in the body of the molded midsole. This enables the EVA midsole to incorporate molded features such as a supportive heel pocket as shown 204, and to better resist degradation from abrasion and from water. This example further illustrates how a degradation sensor such as LC tag 206 can be embedded in a shoe element such as midsole 202, which in this example is further accompanied by embedded conductive element 208.

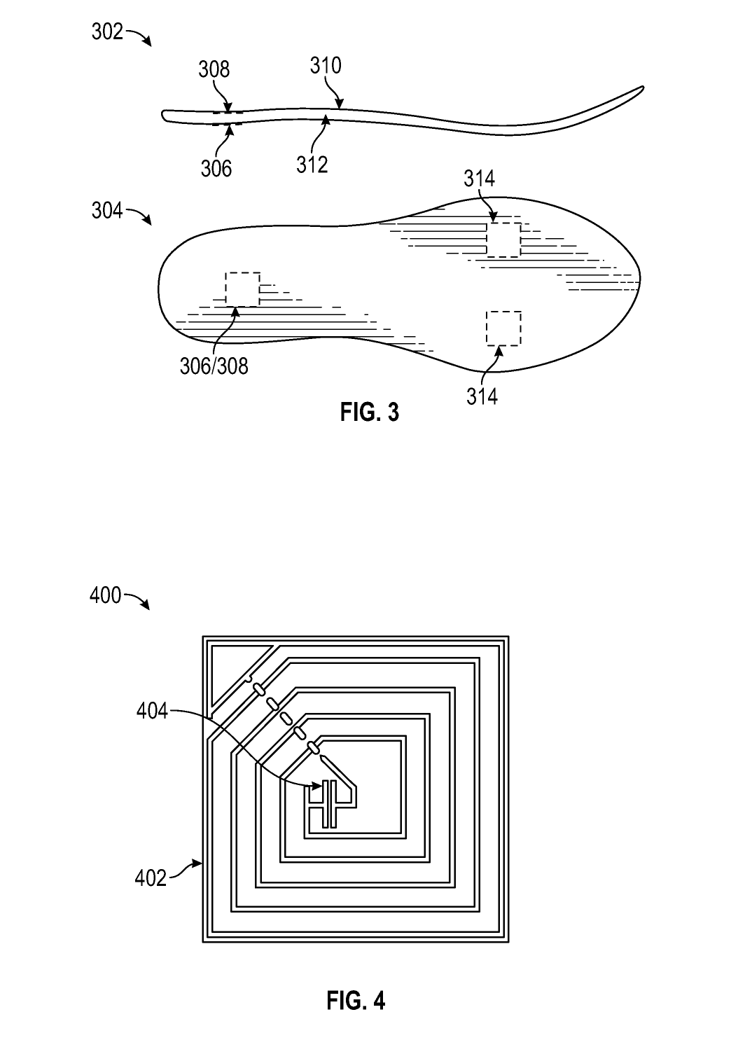

Although the sensors of FIGS. 1 and 2 are embedded within the manufactured shoe, in some embodiments it is desirable for a user to be able to add degradation sensing capabilities to a shoe that is not manufactured with embedded sensors. One example of a sensor configuration designed to address this need is shown in FIG. 3, which illustrates a degradation sensor incorporated into a removable shoe footbed. In FIG. 3, a footbed as shown from the side at 302 and from the top at 304 contains a degradation sensor such as an LC tag 306, which in this example is accompanied by a conductive element 308 attached to the opposite side of the footbed. The LC tag 306 is here attached to the bottom of the footbed such as with an adhesive, and the conductive element 308 is embedded under a fabric layer 310 on top of the footbed but above a foam layer 312 making up the bulk of the footbed's thickness.

The footbed supplied with the shoe can be removed from the foot compartment of the shoe and replaced with the footbed as shown here, such that compression set of the footbed's foam can be determined by measuring the resonant frequency of LC tag 306. Although degradation or compression set of the footbed may indicate when it is time to replace the footbed, in a further example it may further serve as an indication of degradation or compression set to the shoe, as compression set to the footbed and to the shoe will be correlated with one another. In a further example, additional sensors may be integrated into a shoe, such as on or within a midsole, or within a removable footbed as shown at 314.

The degradation sensor in the examples of FIGS. 1-3 comprises an LC tag, such as is shown in FIG. 4. Here, an LC tag shown generally at 100 includes a conductive element that spirals around in a collapsing circular pattern to form an inductive element as shown at 402, which is coupled in parallel with a capacitive element as shown at 404. This inductive element coupled in parallel to a capacitive element forms an LC circuit, which has a resonant frequency based on the capacitance of the capacitive element and the inductance of the inductive element. More specifically, the LC circuit will resonate at

$$f = \frac{1}{2 π \sqrt{LC}}$$

where the frequency f of resonance is determined by the inductance L of the inductive element and the capacitance C of the capacitive element. As the LC tag moves closer to the conductive element in FIGS. 1-3 as a result of increased compression set, the inductance of the inductive element 402 is reduced and the resonant frequency goes up.

The LC tag 400 is formed as a flat element, such as by adhering flat copper traces forming inductive element 402 to a backing material such as paper or plastic film. Capacitor 404 may be a small capacitor coupled in parallel with the inductive element 402, or may be similarly formed of flat copper traces in an alternate embodiment. A typical example LC tag may be an inch square but only a hundredth of an inch thick, such that the relatively flat construction enables the circuit to be easily embedded into or in between layers of a shoe. The relatively large inch square dimensions of the inductive element 402 in the LC circuit make energizing or exciting the LC circuit using external radio frequency source practical, particularly in embodiments where the LC tag is embedded in material or otherwise physically separated by significant distance from the radio frequency energy source.

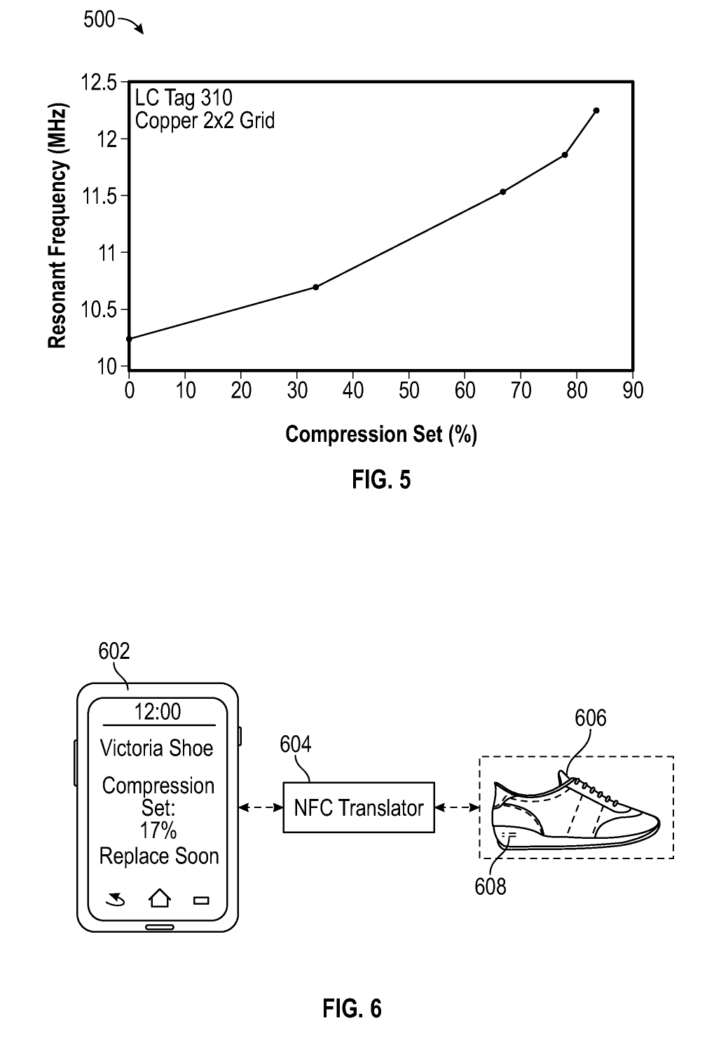

FIG. 5 illustrates how the resonant frequency of an example LC tag degradation sensor varies with compression set. When an LC tag such as that of FIG. 4 is incorporated into a shoe along with a conductive element such as is shown in FIGS. 1 and 2, compression set in the midsole is indicated by an increase in the resonant frequency of the LC tag as a result of a reduction in inductance of the inductive element of the LC tag. The reduction in inductance is a result of the conductive element moving nearer the flat inductive element 402 of the LC tag as shown in FIG. 4, and the conductive element's movement nearer the LC tag when the shoe is at rest is a result of compression set within the midsole of the shoe. As shown in FIG. 5, the LC tag exhibits a resonant peak at approximately 10.25 MHz when the shoe is new and there is no compression set. As the shoe degrades and the midsole undergoes compression set, the resonant frequency increases, such that the resonant frequency at 20% compression set is approximately 10.5 MHz. As shown in the chart, the relationship between compression set and resonant frequency of the LC tag is relatively linear when compression set is low, but becomes less linear as compression set increases. Because a shoe's useful life typically extends only to approximately 20% compression set, estimation of the amount of compression set in a shoe can therefore be accurately estimated using linear interpolation of the relationship between resonant frequency and compression set if the resonant frequencies corresponding to 0% and 20% compression set are known. For example, a shoe having a resonant frequency of 10.375 MHz can be estimated to have 10% compression set, or to have lost approximately 50% of its useful life, using such interpolation.

In other examples, a greater degree of compression set may be observed over the useful life of the material, such as when an LC tag and conductive element are configured to measure compression set in a removable footbed or padded shoe tongue. In such examples, a non-linear curve such as that of FIG. 1 may be used to estimate compression set in the footbed or tongue, as the useful life of the shoe may extend to 50% or greater compression set in the measured shoe element. In an alternate example, several data points are known, and piecewise-linear approximation or other such methods are used between known data points to estimate the compression set based on observed resonant frequency of the LC tag.

FIG. 6 shows an example system for reading the resonant frequency of an LC tag to determine compression set in a shoe. Here, a user device such as a smartphone 602, tablet computer, or personal computer is used to communicate with a reader device such as an NFC translator 604. The NFC translator device 604 is operable to communicate with the user's smartphone 602 using a standard communications technology such as Near-Field Communication (NFC) or Bluetooth, and is also operable to communicate with a shoe 606's degradation sensor 608. In a more detailed example, the NFC translator device 604 includes a resonant frequency detection circuit to query the LC tag that serves as a degradation sensor 608's resonant frequency, such as by broadcasting radio frequency energy across a range of expected resonant frequencies and monitoring for returned energy at the same frequencies. When the NFC translator 604 determines the resonant frequency, it sends this information to the smartphone 602 via a wireless NFC connection, such that the smartphone is operable to receive the resonant frequency information and use known correspondence between observed resonant frequency and compression set as shown in FIG. 5 to present the user with information regarding the useful life of the shoe.

In this example, the smartphone 602 displays to the user the degree of compression set determined to be present in the shoe 606, and the smartphone provides a further indication of what the observed compression set measurement means regarding the useful life of the shoe by indicating that the shoe should be replaced soon. The correspondence between observed compression set to useful life of the shoe is further determined in this example using known characteristics of a particular shoe, which in this example is a Victoria shoe. In other examples, an estimate of useful life may be simply estimated based on compression set data for material typical to a type of shoe, such as EVA compression set in a running shoe.

In alternate examples, the smartphone or other user interface device is operable to provide feedback to a user through other methods, such as using audio, vibration, haptic feedback, or other such methods. The NFC translator 604 in alternate embodiments communicates with the smartphone 602 or other user interface device using wireless communication such as NFC, Bluetooth, ZigBee, WiFi, Cellular, or other wireless communication protocols, or through wired communication such as serial, parallel, USB, analog or digital signal, or other suitable wired communication protocols.

Although the translator device is shown here as being an element separate from the smartphone and the shoe, in other examples it may be integrated into the shoe, integrated into or attached to the smartphone or other user device, or integrated into another device such as a store kiosk or other device providing a user interface.

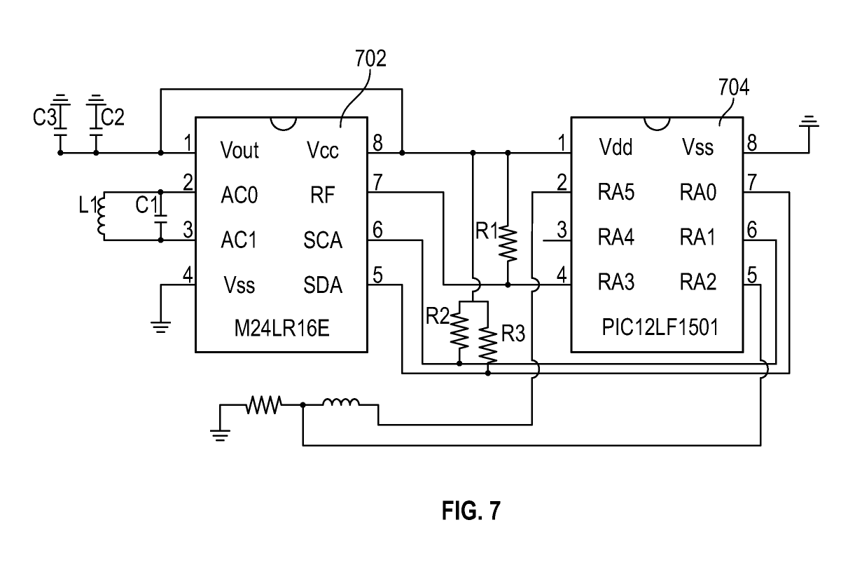

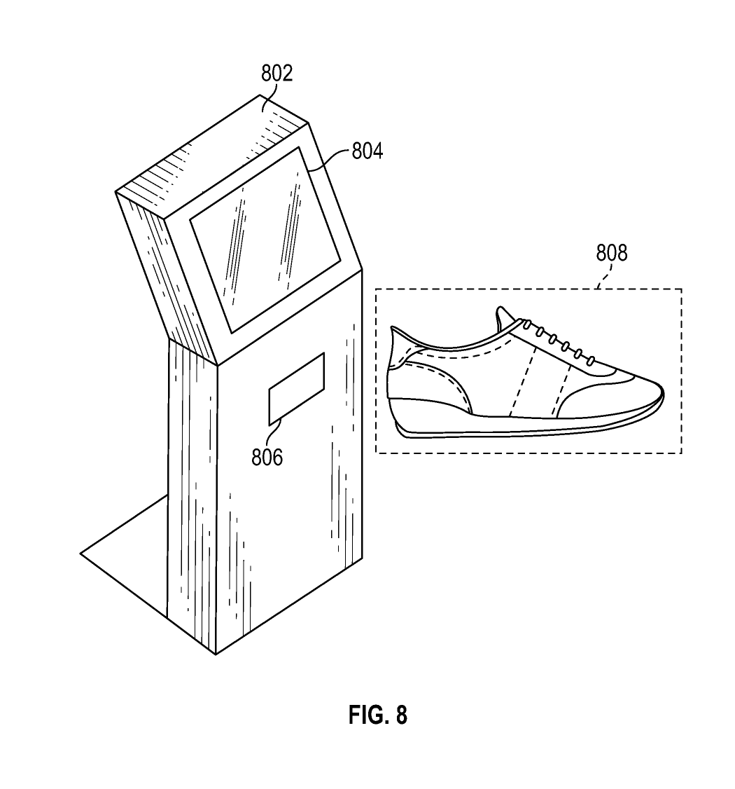

FIG. 7 shows an example translator device circuit operable to query a degradation sensor and provide an indication of the query result to a user device. Here, designator 702 is an ST Microelectronics M24LR16E NFC communications integrated circuit that is operable to communicate with a device such as a smartphone using the NFC protocol. The M24LR16E is further operable to harvest energy through a resonant inductive loop antenna coupled to pins AC0-AC1. Integrated circuit 704 is a Microchip Technology PIC12LF1501 controller that is programmed to communicate with the M24LR integrated circuit, and to interrogate a degradation sensor element such as through an inductive loop coupled to pins RA2-RA5. In operation, a smartphone with an NFC communications module energizes the circuit through radio frequency communication with the resonant inductive loop antenna connected to the M24LR16E, and communicates with the M24LR16E integrated circuit using the wireless NFC protocol. The PIC12LF1501 device provides a variable frequency signal to the inductive loop coupled between pins RA2-RA5, and measures the corresponding current. When the provided frequency matches the resonant frequency of a nearby LC tag the observed current will decrease, indicating the resonant frequency of the LC tag and the corresponding compression set of shoe material as described in prior examples.

A circuit such as that of FIG. 7 may in some embodiments be integrated into a shoe, enabling the shoe to act as an NFC communication device that is operable to communicate directly with a smartphone or other customer device. In one such example, a shoe element incorporating other electronic functions such as accelerometers to measure running distance, pace, or other characteristics incorporates an NFC degradation sensor reader circuit such as that of FIG. 7, and is operable to query the degradation sensor and communicate the sensor data and other information to a nearby device. In other examples, other technologies such as Bluetooth or wired connections are used to couple the degradation sensor to a reader device, or to couple a reader device to a user interface such as a computer, smartphone, or kiosk. The degradation sensor reader circuit such show in FIG. 7 is able to harvest power from the NFC signal provided by a device such as smartphone 602 of FIG. 6, but in other examples will be powered by a battery, by line power, by other power harvesting electronics, through other means, or through a combination thereof.

Although the example translating reader device of FIG. 6 is shown as a standalone device, some examples will incorporate degradation sensor reading elements such as the circuit of FIG. 7 into other configurations external to the shoe. FIG. 8 shows a kiosk incorporating a footwear degradation sensor, consistent with one such example. Here, a kiosk 802, such as may be found standing on the floor in a retail setting, includes a user interface such as a touchscreen display 804, and a shoe degradation sensor reader 806. The kiosk is operable to read information from a degradation sensor such as the LC tag in the examples of FIGS. 1-5 when the shoe is brought near the kiosk's shoe degradation sensor reader 806. Although the kiosk provides many of the same functions as the systems of FIGS. 6-7, it is further operable in various embodiments to perform other functions related to recommendation of new shoes and diagnosis of a user's stride, thereby adding value to the user and for the retailer and shoe manufacturer.

In a more detailed example, a purchaser of new shoes registers the shoes with the kiosk, which takes a baseline or zero compression set measurement for each shoe. The kiosk stores the baseline measurement information in a database or in an account associated with the user, such that the baseline measurement can be easily retrieved later. The user is then able to return with the shoes at a later time to take additional measurements, and the kiosk can determine the degree of degradation to the shoes since they were new using methods such as those described in conjunction with FIGS. 1-6. The kiosk is operable in a further example to read multiple tags per shoe, such as tags on either side of the shoe, or tags in the front and back of the shoe. This provides the kiosk with additional information regarding the relative degradation or compression set of the midsole in different areas of the shoe, enabling the kiosk to characterize the user's stride. The kiosk can then recommend a replacement shoe that might best fit the user's stride based on the degradation or compression set patterns observed in the user's current shoe with use, providing both value to the consumer and the shoe's retailer and manufacturer.

The kiosk in this example is able to characterize the user's stride by taking multiple degradation measurements, such as from different areas of the midsole, and comparing the degradation or compression set observed in the different areas to an expected rate of degradation in each of the areas for a normal stride. If the heel area of the midsole undergoes degradation proportionally faster than the front area of the midsole under the toe box, the kiosk can determine that the runner is a heel striker and may recommend a shoe with more cushioning or less drop from heel to toe. Similarly, if a compression set sensor on one side of a shoe shows proportionally more compression set than a sensor located on the opposite side of the shoe, the kiosk may be able to determine that the user is an overpronator or oversupinator, and recommend a specific shoe having more cushion or more control to address the user's gait characteristics.

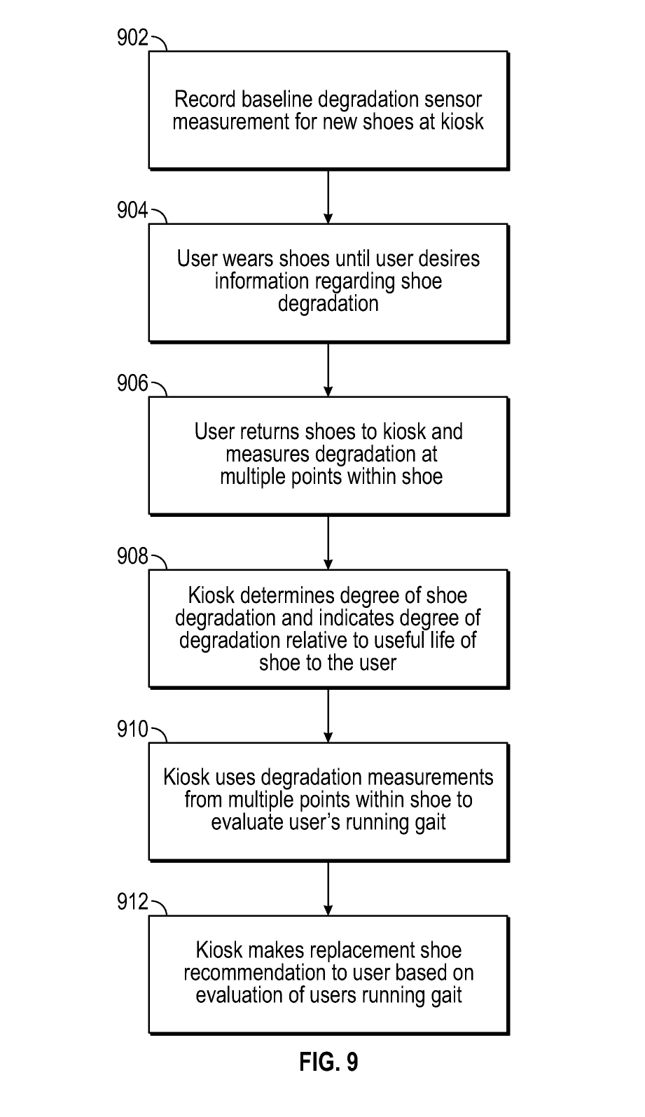

FIG. 9 is a flowchart of a method of using a store kiosk to recommend a replacement shoe to a user. At 902, a user purchases a new pair of shoes, such as running shoes or other athletic shoes, that incorporate degradation sensors. Once the user has decided on a particular pair of shoes, the user records baseline degradation sensor measurement data for the new shoes at the kiosk. This involves in some examples creating a user account with which the shoes can be associated, or in other examples uses a unique machine-readable ID code such as an NFC or RFID tag embedded in the shoe to identify the shoe and associated initial or baseline degradation sensor measurements. In an alternate embodiment, baseline degradation sensor data is based on a known average or characteristic sensor measurement for a particular model of shoe, or is recorded for the shoes as a part of manufacturing or distribution.

Once the baseline data associated with the shoe has been recorded, the user wears the shoes until the user desires information regarding degradation of the shoe at 904. The user then returns to the kiosk at 906, and uses the kiosk to measure the degree of degradation in the shoe. This involves querying one or more sensors in various embodiments, such as LC tags embedded within each side of the shoe, sensors embedded in the toe and heel of the shoe, or both. The kiosk uses the measured degradation information to determine a degree of shoe degradation at 908, and provides an indication to the user of the degree of degradation. In a further example, the degradation is expressed in terms of the measured value, such as a percentage compression set of the midsole of a shoe, or is presented as an estimated percent of useful life left in the shoe. The kiosk in this example is further operable to use degradation measurements from multiple points within the shoe, if available, to evaluate the user's running gait, such as to determine whether the user is a heel striker, overpronates, or oversupinates, at 910. This evaluation can provide valuable feedback for the user regarding their running mechanics and how they may be improved, and can recommend replacement shoes chosen at 912 to address the specific running gait problems identified by evaluating the user's degraded shoes.

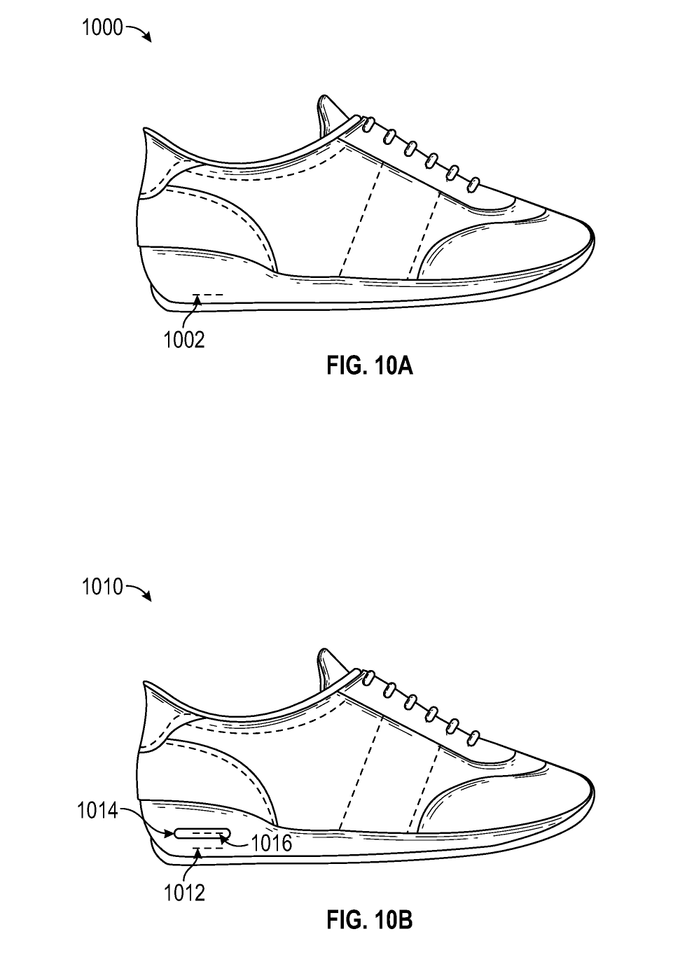

Although many of the examples presented herein use an LC tag and a conductive element to form a degradation sensor, a variety of other degradation sensors are employed in different embodiments. FIG. 10A shows a running shoe incorporating such a degradation sensor. Here, the shoe shown generally at 1000 has a sensor 1002 embedded in the shoe or attached to a portion of the shoe, such that the sensor is operable to indicate a change in at least one physical property of a material in the shoe.

In one more detailed example, the sensor 1002 comprises a thermometer and a heater element, operable to measure thermal conductivity of a shoe element such as an EVA midsole. The heater and thermometer may be separate or may be integrated into the same sensor element, such that a heater operates for a known time and a corresponding increase in temperature is measured at or over a known time to determine how rapidly heat is conducted through the shoe material. The amount of observed temperature rise where the heater and thermometer are located close to one another indicates the specific heat of the material, which can vary with things such as compression set, water absorption, or other forms of degradation. In examples where the heater and thermometer are spaced apart from one another in a shoe material, the observed increase in temperature indicates physical properties such as thermal conductivity or diffusivity of the shoe material, which also vary with degradation such as compression set, water absorption, or the like.

The heater is an example of an active or powered element in that it is electrically or galvanically coupled to a power supply, enabling it to produce heat. The power is supplied via an external reader, via a reader integrated into a shoe, via a battery, or harvested via circuitry configured to capture RF or other energy in various examples. The LC tag incorporated into the degradation in other examples is in contrast a passive sensor, in that it does not contain a power-providing element or amplify power using active electronic devices like transistors or integrated circuits. The LC tag is therefore a passive sensor assembly, whereas a sensor with a heater, integrated circuit, or other such element is considered active. Similarly, the degradation sensors in various embodiments will be operable to provide information via radio frequency energy, such as the LC tag, or via a wired conductive or galvanic connection, such as with a powered heater/thermometer combination. In some such examples, a reader or a power source may be integrated into the shoe, or may be removably integrated into the shoe.

FIG. 10B shows a running shoe incorporating a degradation sensor and a reader. Here, the running shoe shown generally at 1010 incorporates a degradation sensor 1012, and an embedded reader 1014 which is in some embodiments removable. In further examples, the reader 1014 is insertable into and removable from a pocket in the sole of the shoe, such as a pocket under a footbed, in the rear, or in another part of the midsole of the shoe. In still further examples, the reader 1014 is placed in contact with the shoe, such as in contact with the insole, during the measurement and then removed after the measurement. In some examples, the reader is electrically coupled to the degradation sensor 1012 when inserted, while in other examples the reader uses other methods to interact with or take measurements using the degradation sensor.

In a more detailed embodiment of example of FIG. 10B, the degradation sensor comprises an electrically or magnetically conductive element 1012, such as a foil layer, embedded in or near a material layer of the sole. The reader 1014 includes an LC antenna 1016, which can be directly energized via electronics in the reader to sweep across a range of likely resonant frequencies of the LC antenna 1016, which will have a resonant peak indicative of the distance between the LC antenna 1016 and the degradation sensor 1012. As with previous examples, the resonant peak is indicative of the distance between the LC antenna 1016 and the conductive element 1012, such that the reader 1014 can measure and track compression set or other such characteristics in the midsole of the shoe.

Still other sensors will be configured in other examples to measure these or other physical characteristics of various shoe materials, including measuring thickness, compression set, density, elongation, mechanical elasticity, water content, thermal conductivity, electrical conductivity, electrical permittivity, magnetic permeability, and other such characteristics. As various shoe materials degrade with use, it is expected that these and other physical properties will vary in measurable ways, and can be indicated through use of various degradation sensor configurations to determine the degree of degradation of the various materials. For example, a mechanical resonator or vibrator may be able to provide an indication of density, mechanical elasticity, and other such characteristics of a sole material, and an RF coil may be able to provide an indication of electrical conductivity, electrical permittivity, magnetic permeability, or other such characteristics of a material, particularly where the material is embedded with electrically conductive or magnetically conductive particles. Because an increase in particle density will be observable as an increase in electrical or magnetic conductivity, physical properties such as compression set and density can be measured using such methods.

In another example, sensors are further operable to measure a physical property such as the presence of mold, mildew, fungus, bacteria, or other such materials through use of biologic sensors, electrical sensors, or other types of sensor. Indication of physical properties such as these may be incorporated in addition to sensing other physical properties or alone, as a shoe that has not undergone sufficient compression set for a midsole to be worn out may still be discarded if mildew is present within the shoe material.

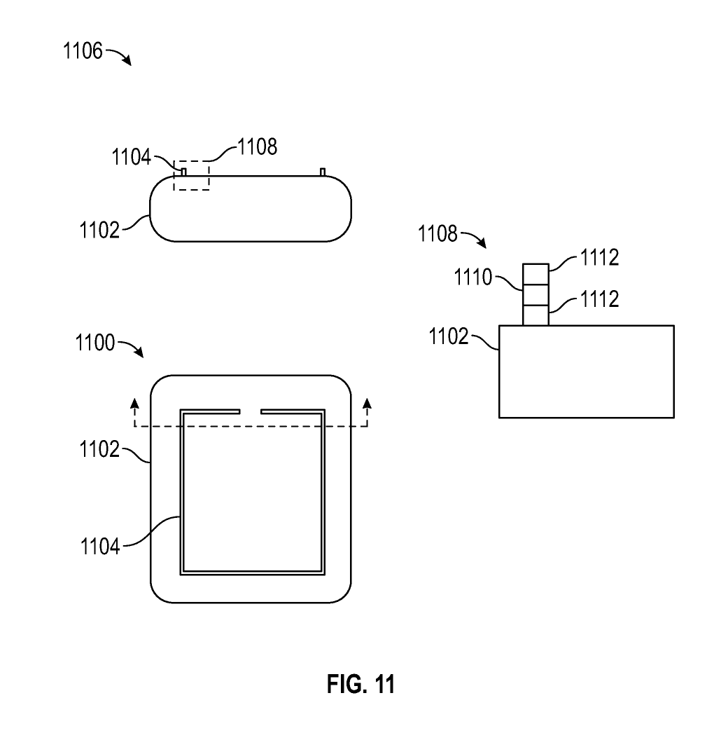

Because water content in a shoe can be an indication of degradation of the shoe material, and can contribute to mold, mildew, and other such material being present in a shoe, it is desirable in some examples to measure water content of shoe material as an indication of degradation of a shoe. FIG. 11 shows a water content shoe degradation sensor. Here, a water content sensor shown generally at 1100 includes a substrate 1102 and an LC resonator 1104. The LC resonator is attached to the surface of the substrate, which can be made of polymer, paper, or any other suitable material. The water content sensor shown at 1100 is shown in side view at 1106, which shows LC resonator 1104 attached to but standing above the substrate 1102. Region 1108 of the plane view shown at 1106 is shown enlarged on the right side of FIG. 11, which more clearly indicates how the LC resonator comprises a dielectric layer 1110 sandwiched between two conductive layers 1112. The dielectric layer 1110 is selected to be a material that absorbs water, such as paper, polymer film, pressure sensitive adhesive, or other material having a relative permittivity much less than that of water. The conductive layers 1112 are made of any suitable conductor, such as copper or other metal, or other conductors.

When the dielectric layer 1110 absorbs water, its relative permittivity goes up, increasing the resulting effective capacitance between the conductive layers 1112. This results in a reduction in resonant frequency of the LC sensor, which can be read using methods such as those described in other examples. In other examples, the LC resonator 1104 may comprise relatively narrower or wider widths, to vary the effective capacitance of the LC resonator or to control resistance in the conductive layers of the LC resonator. In further examples, the path of the LC resonator 1104 on substrate 1102 comprises additional loops, increasing the effective inductance of the LC resonator for various applications. The water sensor of FIG. 11 may be read using an LC tag reader device such as those illustrated and described in conjunction with FIGS. 6-8, or by other methods.

Measurement of material degradation in a shoe is conducted in many examples described herein when the shoe is in a static condition, such as when the shoe not moving and in further examples while the shoe is removed from a user's foot. In other examples, similar degradation measurements may be taken in dynamic conditions, such as when a user is running, walking, jumping, standing on alternating feet, or the like. In dynamic examples such as these, the dynamic condition may be used to measure degradation of the shoe material, such as where running or standing on alternating feet produces a measurable load or impact on the material such that dynamic compression of the foam material or other such physical characteristics can be measured. In further examples, the elastic modulus, viscoelasticity, or force distribution of the shoe are characterized using dynamic measurements, or the dynamic motion is used to determine stride type, to record events such as impacts, strides, steps, or cumulative force encountered by the shoe material. Dynamic activity also enables the degradation sensor, reading apparatus, or other component to harvest energy in some embodiments, such as to power electronics configured to power the degradation sensor, reader, or user interface.

In other examples, sensor measurement may occur during the absence of lower body motion or when the shoes are not being worn. In such examples, shoe degradation monitoring may be based on a change in physical property of the shoe. Key advantages of this measurement method may include simplified and lower cost electronics, sensors, and system design, as reading a static sensor is typically less technically complex than reading a sensor under dynamic conditions. For example, sensor readout time in static examples need not be based on characteristic times of individual motion events, such as strides and jumps. Because many static measures reveal the cumulative effects of dynamic forces over a long measurement or integration time, static measures may provide similarly useful data using lower cost electronics or improved sensitivity relative to dynamic measurements. In addition, static measurement reader electronics are not required to be linked to the shoe's degradation sensors during lower body motion, such as running or jumping, whereas incorporating a dynamic sensor measurement apparatus into the shoe may result in undesirable changes to the shoe mechanics, weight, shoe feel to the user, or increased manufacturing costs. Furthermore, dynamic sensor readings may require robust mechanical connectors, a long range (greater than 5 cm) wireless readout protocol, or measurement error associated with varying human factors or motion styles, contributing to increased cost and decreased reliability of such methods.

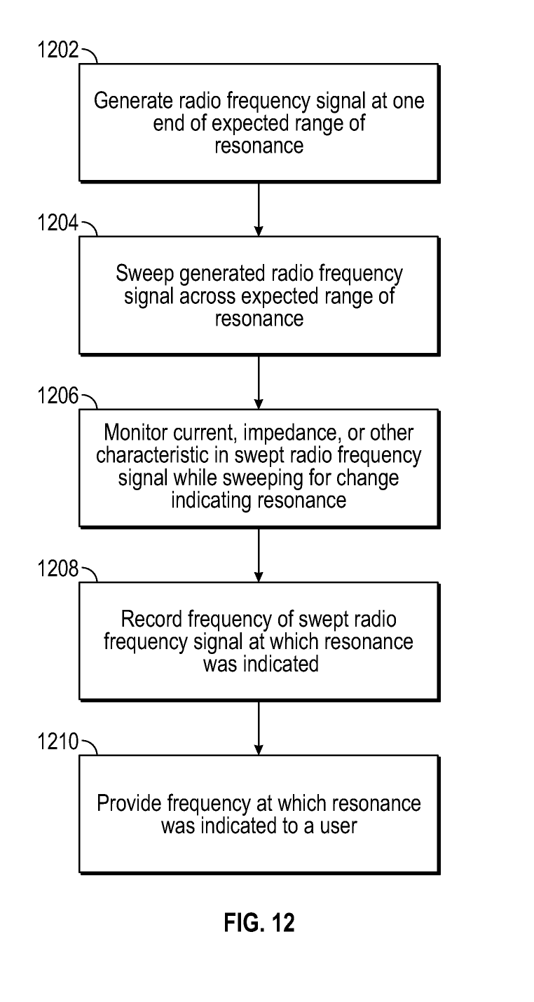

FIG. 12 shows a method of reading a shoe degradation sensor, such as those of FIGS. 1-4 and FIG. 11. At 1202, an electronic circuit generates a radio frequency signal, such as using integrated circuit 704 of FIG. 7. The radio frequency signal sweeps across the expected range of resonance for a degradation sensor incorporating an LC element at 1204, such as in the hundreds of kHz or single digit to tens of MHz range depending on the construction of the LC element. The electronic circuit monitors the current, impedance, or other such characteristic of the swept radio frequency signal at 1206, monitoring for a change such as a decrease in current when the swept frequency matches the resonance of an LC element nearby. When a significant decrease in current or other such change (such as an increase in impedance) is observed, the swept frequency is recorded at 1208. This frequency can then be provided to a user at 1210, stored, or otherwise employed as an indication of the resonant frequency of the LC element, and the physical property of any material configured to influence the inductance or capacitance of the LC element, such as the shoe degradation sensors of FIGS. 1-4 and 11.

Although many of the examples provided herein utilize athletic or running shoes as example footwear, methods and systems similar to these examples may also be applied to a wide range of other footwear, such as casual or dress shoes, hiking or work boots, medical or therapeutic footwear such as diabetic shoes, foot braces, ski boots, skates, socks, compression hosiery, or other such footwear.

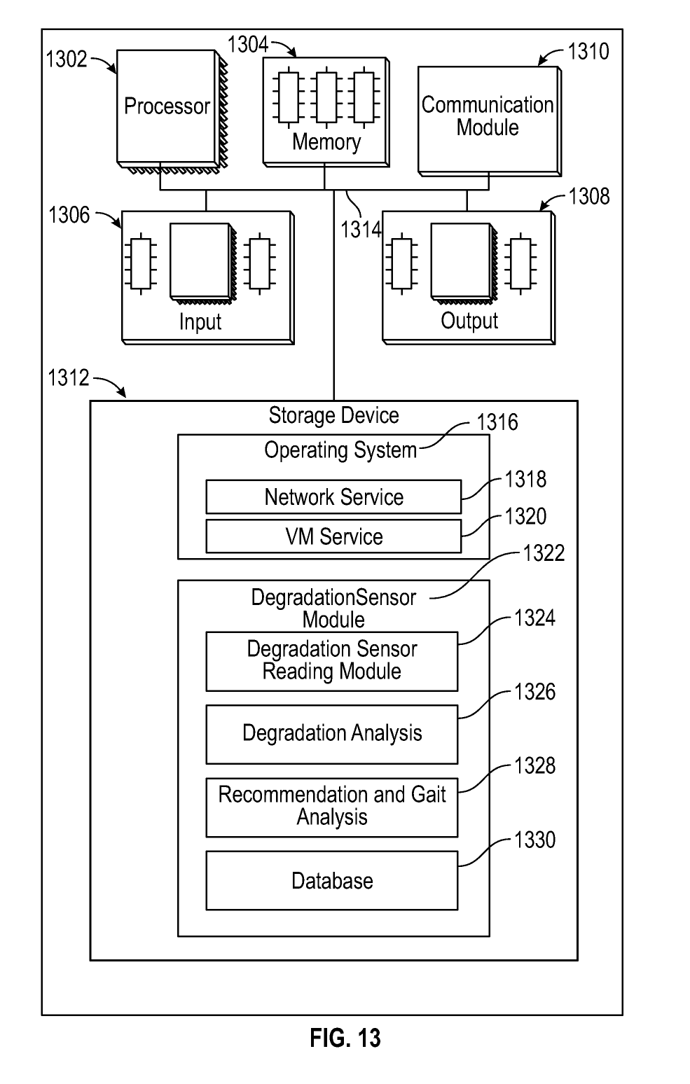

The method of FIG. 12 may be implemented in part using a computerized device, such as a smartphone, kiosk, or other computerized device. Similarly, many of the other methods described herein or parts of such methods, such as recording baseline degradation sensor information for new shoes, can be performed using a computerized system. FIG. 13 shows a computerized shoe degradation sensor measurement system, consistent with various examples described herein. FIG. 13 illustrates only one particular example of computing device 1300, and other computing devices 1300 may be used in other embodiments. Although computing device 1300 is shown as a standalone computing device, computing device 1300 may be any component or system that includes one or more processors or another suitable computing environment for executing software instructions in other examples, and need not include all of the elements shown here.

As shown in the specific example of FIG. 13, computing device 1300 includes one or more processors 1302, memory 1304, one or more input devices 1306, one or more output devices 1308, one or more communication modules 1310, and one or more storage devices 1312. Computing device 1300, in one example, further includes an operating system 1316 executable by computing device 1300. The operating system includes in various examples services such as a network service 1318 and a virtual machine service 1320 such as a virtual server. One or more applications, such as a degradation sensor software module 1322 are also stored on storage device 1312, and are executable by computing device 1300.

Each of components 1302, 1304, 1306, 1308, 1310, and 1312 may be interconnected (physically, communicatively, and/or operatively) for inter-component communications, such as via one or more communications channels 1314. In some examples, communication channels 1314 include a system bus, network connection, inter-processor communication network, or any other channel for communicating data. Applications such as recommendation module 1322 and operating system 1316 may also communicate information with one another as well as with other components in computing device 1300.

Processors 1302, in one example, are configured to implement functionality and/or process instructions for execution within computing device 1300. For example, processors 1302 may be capable of processing instructions stored in storage device 1312 or memory 1304. Examples of processors 1302 include any one or more of a microprocessor, a controller, a digital signal processor (DSP), an application specific integrated circuit (ASIC), a field-programmable gate array (FPGA), or similar discrete or integrated logic circuitry.

One or more storage devices 1312 may be configured to store information within computing device 1300 during operation. Storage device 1312, in some examples, is known as a computer-readable storage medium. In some examples, storage device 1312 comprises temporary memory, meaning that a primary purpose of storage device 1312 is not long-term storage. Storage device 1312 in some examples is a volatile memory, meaning that storage device 1312 does not maintain stored contents when computing device 1300 is turned off. In other examples, data is loaded from storage device 1312 into memory 1304 during operation. Examples of volatile memories include random access memories (RAM), dynamic random access memories (DRAM), static random access memories (SRAM), and other forms of volatile memories known in the art. In some examples, storage device 1312 is used to store program instructions for execution by processors 1302. Storage device 1312 and memory 1304, in various examples, are used by software or applications running on computing device 1300 such as recommendation module 1322 to temporarily store information during program execution.

Storage device 1312, in some examples, includes one or more computer-readable storage media that may be configured to store larger amounts of information than volatile memory. Storage device 1312 may further be configured for long-term storage of information. In some examples, storage devices 1312 include non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

Computing device 1300, in some examples, also includes one or more communication modules 1310. Computing device 1300 in one example uses communication module 1310 to communicate with external devices via one or more networks, such as one or more wireless networks. Communication module 1310 may be a network interface card, such as an Ethernet card, an optical transceiver, a radio frequency transceiver, or any other type of device that can send and/or receive information. Other examples of such network interfaces include Bluetooth, 3G or 4G, WiFi radios, and Near-Field Communications (NFC), and Universal Serial Bus (USB). In some examples, computing device 1300 uses communication module 1310 to wirelessly communicate with an external device such as via public network such as the Internet.

Computing device 1300 also includes in one example one or more input devices 1306. Input device 1306, in some examples, is configured to receive input from a user through tactile, audio, or video input. Examples of input device 1306 include a touchscreen display, a mouse, a keyboard, a voice responsive system, video camera, microphone or any other type of device for detecting input from a user.

One or more output devices 1308 may also be included in computing device 1300. Output device 1308, in some examples, is configured to provide output to a user using tactile, audio, or video stimuli. Output device 1308, in one example, includes a display, a sound card, a video graphics adapter card, or any other type of device for converting a signal into an appropriate form understandable to humans or machines. Additional examples of output device 1308 include a speaker, a light-emitting diode (LED) display, a liquid crystal display (LCD), or any other type of device that can generate output to a user.

Computing device 1300 may include operating system 1316. Operating system 1316, in some examples, controls the operation of components of computing device 1300, and provides an interface from various applications such as degradation sensor software module 1322 to components of computing device 1300. For example, operating system 1316, in one example, facilitates the communication of various applications such as degradation sensor software module 1322 with processors 1302, communication unit 1310, storage device 1312, input device 1306, and output device 1308. Applications such as degradation sensor software module 1322 may include program instructions and/or data that are executable by computing device 1300. As one example, degradation sensor software module 1322 and its degradation sensor reading module 1324, degradation analysis module 1326, recommendation and gait analysis module 1328, and database 1330 may include instructions that cause computing device 1300 to perform one or more of the operations and actions described in the examples presented herein.

### EXEMPLARY EMBODIMENTS

### Embodiment 1

A shoe degradation sensor assembly, comprising:

a material layer between a foot space and an outer surface of a shoe;

a first sensor disposed in or proximate to the material layer of the shoe, wherein the material layer changes in at least one physical property with degradation to the shoe, the first sensor configured to indicate the at least one physical property of the material layer thereby indicating a degree of degradation to the shoe.

### Embodiment 2

The electrically-readable shoe degradation sensor assembly of Embodiment 1, wherein the first sensor is configured to measure the at least one physical property of the shoe under static conditions.

### Embodiment 3

The electrically-readable shoe degradation sensor assembly of Embodiment 1 or 2, wherein the first sensor is configured to emulate a passive near-field communication (NFC) tag.

### Embodiment 4

The electrically-readable shoe degradation sensor assembly of any one of Embodiments 1-3, wherein the first sensor comprises an LC (inductor-capacitor) network having a resonant property affected by the at least one physical property.

### Embodiment 5

The electrically-readable shoe degradation sensor assembly of Embodiment 4, further comprising at least one conductive or magnetic element spaced apart from the LC network, such that degradation to material between the conductive or magnetic element and LC network affects at least one of resonant frequency and quality factor of the LC network.

### Embodiment 6

The electrically-readable shoe degradation sensor assembly of Embodiment 5, wherein the resonant property affected by the at least one physical property comprises at least one of resonant frequency and quality factor.

### Embodiment 7

The electrically-readable shoe degradation sensor assembly of Embodiment 4, wherein at least a portion of the LC network is contained within a radio frequency identification tag.

### Embodiment 8

The electrically-readable shoe degradation sensor assembly of any one of Embodiments 1-7, wherein the first sensor comprises at least one of an electrically conductive or magnetically conductive layer configured to interact with an electromagnetic field of a reader.

### Embodiment 9

The electrically-readable shoe degradation sensor assembly of Embodiment 8, wherein the electrically conductive (or magnetic) element is embedded in the material layer.

### Embodiment 10

The electrically-readable shoe degradation sensor assembly Embodiment 8, wherein the electrically conductive (or magnetic) element is disposed on the material layer.

### Embodiment 11

The electrically-readable shoe degradation sensor assembly of any one of Embodiments 1-10, wherein the physical property comprises at least one of thickness, compression set, density, elongation, mechanical elasticity, water content, thermal conductivity, electrical conductivity, electrical permittivity, magnetic permeability, presence of fungus, presence of mildew, presence of bacteria, and presence of mold.

### Embodiment 12

The electrically-readable shoe degradation sensor assembly of any one of Embodiments 1-11, wherein the physical property comprises a presence of at least one of fungus, mildew, bacteria, and mold.

### Embodiment 13

The electrically-readable shoe degradation sensor assembly of any one of Embodiments 1-12, wherein the first sensor comprises a mechanical resonator.

### Embodiment 14

The electrically-readable shoe degradation sensor assembly of any one of Embodiments 1-13, wherein the first sensor comprises a thermal sensor.

### Embodiment 15

The electrically-readable shoe degradation sensor assembly of Embodiment 14, further comprising a heater, such that the thermal sensor's response to actuation of the heater indicates the at least one physical property of the material layer.

### Embodiment 16

The electrically-readable shoe degradation sensor assembly of Embodiment 15, wherein the heater and thermal sensor are the same element.

### Embodiment 17

The electrically-readable shoe degradation sensor assembly of any one of Embodiments 1-16, further comprising at least one of a removable insole, midsole, and wedge element comprising the material layer.

### Embodiment 18

The electrically-readable shoe degradation sensor assembly of Embodiment 1, wherein the material layer comprises a midsole or wedge element of the shoe.

### Embodiment 19

The electrically-readable shoe degradation sensor assembly of any one of Embodiments 1-18, wherein an absolute measurement indicates the at least one physical property of the material layer, thereby indicating a degree of degradation to the shoe.

### Embodiment 20

The electrically-readable shoe degradation sensor assembly of any one of Embodiments 1-19, wherein measurement from the sensor relative to a reference measurement indicates the change in the at least one physical property of the material layer, thereby indicating a degree of degradation to the shoe.

### Embodiment 21

The electrically-readable shoe degradation sensor assembly of any one of Embodiments 1-20, wherein the material layer is comprised of a composite material.

### Embodiment 22

The electrically-readable shoe degradation sensor assembly of any one of Embodiments 1-21, wherein the material layer comprises a fluid-filled element.

### Embodiment 23

The electrically-readable shoe degradation sensor assembly of any one of Embodiments 1-22, wherein the material layer is constructed to store mechanical energy.

### Embodiment 24

The electrically-readable shoe degradation sensor assembly of any one of Embodiments 1-23, wherein the material layer contains at least one of ethylene vinyl acetate, polyurethane, polymeric foam, rubber, nylon, fabric, gel, adhesive, polychloroprene, thermoplastic resin, thermoset resin, and air.

### Embodiment 25

The electrically-readable shoe degradation sensor assembly of any one of Embodiments 1-24, wherein the electrically-readable shoe degradation sensor is electrically readable through a galvanic coupling between the sensor and a reader.

### Embodiment 26

The electrically-readable shoe degradation sensor assembly of any one of Embodiments 1-25, wherein the electrically-readable shoe degradation sensor is electrically readable via a wireless reader.

### Embodiment 27

The shoe degradation sensor assembly of Embodiment 1, wherein the first sensor comprises a passive sensor device.

### Embodiment 28

A method of determining shoe degradation, comprising:

measuring at least one physical property of a material layer between a foot space and an outer surface of a shoe using a first sensor embedded in or proximate to a material layer, wherein the physical property of the material layer changes with degradation to the shoe, the measurement thereby indicating a degree of degradation to the shoe.

### Embodiment 29

The method of determining shoe degradation of Embodiment 28, wherein measuring the at least one physical property of the material layer comprises querying the first sensor embedded within or proximate to the material layer.

### Embodiment 30

The method of determining shoe degradation of Embodiment 29, wherein measuring the at least one physical property of the material layer comprises further querying a second sensor embedded within or proximate to the material layer.

### Embodiment 31

The method of determining shoe degradation of Embodiment 29 or 30, wherein the first sensor comprises a LC network, and measurement comprises measuring at least one resonant property of the LC circuit.

### Embodiment 32

The method of determining shoe degradation of any one of Embodiments 29-31, wherein the first sensor comprises a conductive or magnetic layer configured to interact with an electromagnetic field of a reader, and measurement comprises measuring interaction of the layer with the electromagnetic field of the reader

### Embodiment 33

The method of determining shoe degradation of any one of Embodiments 29-32, wherein the first sensor is configured to communicate as a near-field communication (NFC) tag.

### Embodiment 34

The method of determining shoe degradation of any one of Embodiments 29-33, wherein the physical property comprises at least one of thickness, compression set, density, elongation, mechanical elasticity, water content, thermal conductivity, electrical conductivity, electrical permittivity, and magnetic permeability.

### Embodiment 35

The method of determining shoe degradation of any one of Embodiments 29-34, wherein the first sensor is a passive sensor.

### Embodiment 36

The method of determining shoe degradation of any one of Embodiments 29-35, wherein querying the first sensor comprises establishing radio frequency (RF) communication with the sensor.

### Embodiment 37

The method of determining shoe degradation of any one of Embodiments 28-36, further comprising comparing the measurement of the at least one physical property of the shoe to a reference measurement to estimate a degree of degradation to the shoe.

### Embodiment 38

The method of determining shoe degradation of any one of Embodiments 28-37, further comprising presenting the indicated degree of shoe degradation to a user.

### Embodiment 39

A shoe degradation measurement system, comprising:

a shoe having a first sensor embedded in or proximate to a material layer of the shoe between a foot space and an outer surface of the shoe, wherein the material layer changes in at least one physical property with degradation to the shoe, the first sensor configured to indicate the at least one physical property of the material layer thereby indicating a degree of degradation to the shoe;

a computerized system configured to provide an indication of shoe degradation to a user based on a measurement of the at least one physical property of the material from the first sensor; and

an interface coupled to the computerized system and configured to receive from the first sensor an indication of the measurement of the at least one physical property of the material from the first sensor.

### Embodiment 40

The shoe degradation measurement system of Embodiment 39, wherein the interface comprises a near-field communication (NFC) tag reader.

### Embodiment 41

The shoe degradation measurement system of Embodiment 39 or 40, wherein the interface is located physically near but not in contact with the shoe when it receives from the first sensor an indication of the measurement of the at least one physical property of the material from the first sensor.

### Embodiment 42

The shoe degradation measurement system of any one of Embodiments 39-41, wherein the interface is galvanically coupled to the first sensor when it receives from the first sensor an indication of the measurement of the at least one physical property of the material from the first sensor.

### Embodiment 43

The shoe degradation measurement system of any one of Embodiments 39-42, wherein the interface is integrated into the computerized system.

### Embodiment 44

The shoe degradation measurement system of any one of Embodiments 39-43, wherein the computerized system and interface comprise part of a kiosk.

### Embodiment 45

The shoe degradation measurement system of any one of Embodiments 39-44, wherein the computerized system comprises part of a smartphone.

### Embodiment 46

The shoe degradation measurement system of any one of Embodiments 39-45, wherein the computerized system is further configured to diagnose a gait of a user who has used the shoe based on the measurement of the at least one physical property of the material from the first sensor.

### Embodiment 47

The shoe degradation measurement system of any one of Embodiments 39-46, wherein the computerized system is further configured to recommend a specific replacement shoe having specific characteristics for a user who has used the shoe based on the measurement of the at least one physical property of the material from the first sensor.

### Embodiment 48

The shoe degradation measurement system of any one of Embodiments 39-47, wherein the computerized system is further configured to store baseline data for a shoe based on the measurement of the at least one physical property of the material from the first sensor, such that the baseline data can be compared with subsequent measurement of the at least one physical property of the material from the first sensor to estimate degradation.

### Embodiment 49

The shoe degradation measurement system of any one of Embodiments 39-48, wherein the computerized system is operable to store the baseline data in a database associated with an electronic identifying tag incorporated into the shoe.

### Embodiment 50

The shoe degradation measurement system of any one of Embodiments 39-49, wherein the computerized system is operable to store the baseline data in an electronic device associated with the shoe.

### Embodiment 51

A shoe degradation measurement reader, comprising:

an electronic device configured to query a first sensor disposed in or proximate to a material layer of a shoe between a foot space and an outer surface of the shoe, wherein the material layer changes in at least one physical property with degradation to the shoe, the first sensor configured to indicate the at least one physical property of the material layer thereby indicating a degree of degradation to the shoe.

### Embodiment 52

The shoe degradation measurement reader of Embodiment 51, wherein the electronic device comprises a near-field communication (NFC) tag reader and the first sensor comprises a sensor configured to operate as an NFC tag.

### Embodiment 53

The shoe degradation measurement reader of Embodiment 51 or 52, wherein the electronic device comprises a resonant property measurement circuit, and the first sensor comprises a sensor configured to change in at least one resonant property with a change in the at least one physical property of the shoe.

### Embodiment 54

The shoe degradation measurement reader of any one of Embodiments 51-53, wherein the electronic device generates an electromagnetic field, and the sensor comprises an electrically conductive element disposed in or proximate to the material layer, and the element is configured to modify the generated electromagnetic field with a change in the at least one physical property of the shoe.

### Embodiment 55

The shoe degradation measurement reader of any one of Embodiments 51-54, wherein the electronic device comprises a thermal measurement apparatus operable to heat a heater disposed in contact with the material layer of the shoe and to measure temperature via the first sensor, wherein the sensor comprises a thermal sensor.

### Embodiment 56

The shoe degradation measurement reader of any one of Embodiments 51-55, wherein the electronic device comprises a conductance meter operable to measure conductance between the first sensor and a second sensor disposed within the material layer of the shoe.

### Embodiment 57

The shoe degradation measurement reader of any one of Embodiments 51-56, wherein the electronic device comprises a radio frequency device operable to communicate with at least the first sensor.

### Embodiment 58

The shoe degradation measurement reader of any one of Embodiments 51-57, wherein the electronic device is attached to the shoe.

### Embodiment 59

The shoe degradation measurement reader of any one of Embodiments 51-58, wherein the electronic device is operable to query sensors disposed in different shoes.

### Embodiment 60

The shoe degradation measurement reader of any one of Embodiments 51-59, wherein the electronic device is powered by a battery, line power, power harvesting electronics, or a combination thereof.

### Embodiment 61

The shoe degradation measurement reader of any one of Embodiments 51-60, wherein the electronic device communicates with a user interface device via one or more wired (serial, parallel, analog) or wireless (NFC, RFID, Bluetooth, Zigbee, WiFi, Cellular) interfaces.

### Embodiment 62

The shoe degradation measurement reader of Embodiment 61, wherein the user interface device comprises at least part of a smartphone, tablet, kiosk, display.

### Embodiment 63

The shoe degradation measurement reader of any one of Embodiments 51-62, wherein the electronic device comprises a user interface device.

Although specific embodiments have been illustrated and described herein, any arrangement that achieve the same purpose, structure, or function may be substituted for the specific embodiments shown. This application is intended to cover any adaptations or variations of the example embodiments of the invention described herein. These and other embodiments are within the scope of the following claims and their equivalents.

1. A shoe degradation sensor assembly, comprising:
a material layer between a foot space and an outer surface of a shoe;
a first sensor disposed in or proximate to the material layer of the shoe, wherein the material layer changes in at least one physical property with degradation to the shoe, the first sensor configured to indicate the at least one physical property of the material layer thereby indicating a degree of degradation to the shoe.

2. The electrically-readable shoe degradation sensor assembly of claim 1, wherein the first sensor is configured to measure the at least one physical property of the shoe under static conditions.

3. The electrically-readable shoe degradation sensor assembly of claim 1, wherein the first sensor comprises an LC (inductor-capacitor) network having a resonant property affected by the at least one physical property.

4. The electrically-readable shoe degradation sensor assembly of claim 3, further comprising at least one of a conductive element and a magnetic element spaced apart from the LC network, such that degradation to material between the conductive or magnetic element and LC network affects at least one of resonant frequency and quality factor of the LC network.

5. The electrically-readable shoe degradation sensor assembly of claim 1, wherein the first sensor comprises at least one of an electrically conductive layer and a magnetically conductive layer, configured to interact with an electromagnetic field of a reader.

6. The electrically-readable shoe degradation sensor assembly of claim 1, wherein the physical property comprises at least one of thickness, compression set, density, elongation, mechanical elasticity, water content, thermal conductivity, electrical conductivity, electrical permittivity, magnetic permeability, presence of fungus, presence of mildew, presence of bacteria, and presence of mold.

7. The electrically-readable shoe degradation sensor assembly of claim 1, further comprising a heater, wherein the first sensor comprises a thermal sensor, and wherein the thermal sensor's response to actuation of the heater indicates the at least one physical property of the material layer.

8. The electrically-readable shoe degradation sensor assembly of claim 1, further comprising at least one of a removable insole, midsole, and wedge element comprising the material layer.

9. The shoe degradation sensor assembly of claim 1, wherein the first sensor comprises a passive sensor.

10. The shoe degradation sensor assembly of claim 1, further comprising a second sensor disposed in or proximate to the material layer of the shoe, wherein the second sensor is a location different from a location of the first sensor.

11. A shoe degradation measurement system, comprising:
a shoe having a first sensor embedded in or proximate to a material layer of the shoe between a foot space and an outer surface of the shoe, wherein the material layer changes in at least one physical property with degradation to the shoe, the first sensor configured to indicate the at least one physical property of the material layer thereby indicating a degree of degradation to the shoe;
a computerized system configured to provide an indication of shoe degradation to a user based on a measurement of the at least one physical property of the material from the first sensor; and
an interface coupled to the computerized system and configured to receive from the first sensor an indication of the measurement of the at least one physical property of the material from the first sensor.

12. The shoe degradation measurement system of claim 11, wherein when the interface receives from the first sensor the indication of the measurement of the at least one physical property of the material from the first sensor, the interface is located physically proximate to the shoe.

13. The shoe degradation measurement system of claim 11, wherein when the interface receives from the first sensor the indication of the measurement of the at least one physical property of the material from the first sensor, the interface is galvanically coupled to the first sensor.

14. The shoe degradation measurement system of claim 11, wherein the computerized system is further configured to diagnose a gait of a user who has used the shoe based on the measurement of the at least one physical property of the material from the first sensor.

15. The shoe degradation measurement system of claim 11, wherein the computerized system is further configured to recommend a replacement shoe having specific characteristics for a user who has used the shoe based on the measurement of the at least one physical property of the material from the first sensor.
